(19) United States
(12) Patent Application Publication
(10) Pub. No.: US 2026/0363989 A1
(43) Pub. Date: Nov. 19, 2026

(54) BOTULINUM TOXIN INJECTION IN PLATYSMA FOR LOWER FACE AND NECK REJUVENATION

(71) Applicant: Merz Pharma GmbH & Co. KGaA, Frankfurt am Main (DE)

(72) Inventors: Gemma Odena, Raleigh, NC (US); Ross Radusky, Dallas, TX (US); Susanna Roll, Stadecken-Elsheim (DE); Petra Weissenberger, Frankfurt am Main (DE); Vladimir Sudimac, Frankfurt am Main (DE); Iryna Heide, Frankfurt am Main (DE)

(21) Appl. No.: 19/132,547

(22) PCT Filed: Nov. 27, 2023
(86) PCT No.: PCT/EP2023/083214

§ 371 Date: May 23, 2025

(30) Foreign Application Priority Data
Nov. 28, 2022 (EP) 22210037.2

(51) Int. Cl. A61K 8/66; A61Q 19/08

(52) U.S. Cl. CPC A61K 8/66; A61Q 19/08; A61K 2800/91; C12Y 304/24069

(57) ABSTRACT

The present invention generally relates to the use of botulinum toxin for lower face and neck rejuvenation by injection of highly concentrated botulinum toxin into platysma, more specifically into platysma bands and the platysma muscle at the jawline. Specifically, the botulinum toxin is injected in the platysma bands and the platysma muscle at the jawline at a dose of 3 U to 5 U and a volume of 0.04 ml to 0.06 ml per injection point. Furthermore, the present invention relates to a method for lower face and neck rejuvenation comprising injection of botulinum toxin in platysma bands and in the platysma muscle at the jawline at a dose of 3 U to 5 U and a volume of 0.04 ml to 0.06 ml per injection point.

### FIELD OF THE INVENTION

The present invention generally relates to the use of botulinum toxin for lower face and neck rejuvenation by injection of highly concentrated botulinum toxin into platysma, more specifically into platysma bands and the platysma muscle at the jawline. Specifically, the botulinum toxin is injected in the platysma bands and the platysma muscle at the jawline at a dose of 3 U to 5 U and a volume of 0.04 ml to 0.06 ml per injection point. Furthermore, the present invention relates to a method for lower face and neck rejuvenation comprising injection of botulinum toxin in platysma bands and in the platysma muscle at the jawline at a dose of 3 U to 5 U and a volume of 0.04 ml to 0.06 ml per injection point.

### BACKGROUND OF THE INVENTION

Restoring volume to the mid-face and treating wrinkles in the upper face have been the focus of facial rejuvenation for many years. However, over the past few years, the lower face and the neck have become the focus of facial rejuvenation, especially in the higher age group. A sharp, defined jawline is associated with a youthful appearance and a slender, streamlined neck is perceived as a sign of health and youth. With aging, however, the jawline loses its sharpness resulting in a less defined or sagging jawline and the neck begins to show platysma bands, sagging skin, and prominent folds or jowls.

The platysma is a thin sheet of muscle that envelopes the entire neck. It arises from the fascia that covers the upper segments of the deltoid and pectoralis muscles. Its thin muscle fibers cross over the clavicle and proceed obliquely superiorly, laterally and medially over the neck. The platysma crosses the mandible, with some fibers being inserted into the bone below the oblique line and others extending approximately 4 cm above the mandibular border into the cheek and inserting into the skin and subcutaneous tissue. In addition, some muscle fibers insert in the mentalis muscle and the depressor anguli oris.

In terms of function, the platysma is a mimetic muscle that contributes to facial expression. It distends the skin of the neck and pulls it downward. Laterally, it acts in synergy with the depressor anguli oris (DAO) and lowers the corner of the lower lips by pulling down the lateral corner of the mouth. It is also involved in the formation of transversal lines of the neck. Thus, contraction of the platysma pulls down the mandible, the corner of the mouth and the lower lip. During the aging process the muscle begins to separate and bunch into cords or bands usually in the midline para median at each side of the neck, but also at the side of the neck. These cords or bands are called platysma bands (or platysmal bands) and are characteristic of an aging neck.

A variety of surgical techniques for treating platysma bands are used to improve or restore the definition of the neck. However, unfavorable surgical outcomes and the understanding that platysma bands are caused by muscular activity during the aging process (see, e.g., Trévidic, P. and Criollo-Lamilla, G., Plast. Reconstr. Surg. 2017; 139 (1): 41-47) have shifted treatment towards non-surgical methods like injections of botulinum toxin. Botulinum toxin is a neurotoxin from Clostridium botulinum that blocks the release of acetylcholine into the synaptic cleft, thereby preventing cholinergic neuromuscular innervation. It exists in different serotypes (types A-H) and is produced as non-covalent macromolecular protein complexes consisting of the active 150 kDa neurotoxin and nontoxic neurotoxin-associated proteins (NAPs or non-toxic “complexing proteins”). The 150 kDa neurotoxin is synthesized as single chain polypeptide that is proteolytically cleaved into two chains, i.e., a 50 kDa light chain and a 100 kDa heavy chain, which are connected by a disulfide bond.

Botulinum toxin can relax and flatten the platysma bands to make the bands less pronounced. It also improves transversal (horizontal) neck lines. Furthermore, by reducing the degree of downward pull, injection of the platysma can also be used in the treatment of the lower face contour, e.g., the contour of the jawline, for global facial rejuvenation. In addition, the reduced downward pull of botulinum toxin-injected platysma can improve lateral cheek lines and marionette lines, lifting the lower face (see, e.g., Tamura, B. M., The Effect of Botulinum Toxin on the Platysma Muscle, Curr. Derm. Rep. 2012, 1:89-95; Kane et al., Expanding the Use of Neurotoxins in Facial Aesthetics: A Consensus Panel's Assessment and Recommendations, J. Drugs Dermatol. 2010, 9 (1):s7-s25).

However, the use of botulinum toxin in the lower face and neck is more complex than its use in the upper face and, thus, fraught with more complications. This is because the muscles of this region are very close together and interface at different levels and depths to perform heterogeneous functions, such as talking, eating, drinking, and expressivity of the face. Injected botulinum toxin may spread or diffuse to muscles of deglutition, the pharynx and neck flexors structures and cause complications like swallowing difficulties (dysphagia), dysphonia, and neck weakness (see, e.g., De Boulle et al., Chapter 15: Neck Rejuvenation, in: Botulinum Toxins: Cosmetic and Clinical Applications, Cohen, J. L., Ozog, D. M., and Porto, D. A. (eds.), Wiley-Blackwell, 2017; Chen, D. L. and Cohen, J. L., Botulinum Toxin-A Chemical Denervation for Platysmal Bands: Maximal Dosing Considerations, J. Drugs Dermatol. 2015, 14(9):931; Carruthers et al., Consensus Recommendations on the Use of Botulinum Toxin Type A in Facial Aesthetics, Plast. Reconstr. Surg. 2004, 114(6 Suppl):1S-22S)).

These undesirable side effects can be reduced by using lower doses. However, low doses are less effective and may lead to residual banding and development of compensatory bands due to incomplete paralysis of the platysma muscle. Also, when using low doses, the duration of effect is shorter and more frequent treatments are needed. The use of low volumes may also result in less side effects due to a decreased spread, but at the same time the toxin dose may be too low to result in an efficient treatment.

In attempts to reduce undesirable side effects while achieving high treatment efficacy, the impact of toxin dilution (concentration, volume) has been studied, but with controversial results. On the one hand, it was found that dilution increases the incidence of adverse effects observed in the treatment of glabellar rhytides, and had no significant effect on efficacy or duration (Carruthers et al., Dilution volume of botulinum toxin type A for the treatment of glabellar rhytides: does it matter?, Dermatol. Surg. 2007, 33:S97-S104). On the other hand, it was found that larger volumes are beneficial in the treatment of dynamic forehead lines, requiring fewer injections and thereby reducing injection pain and increasing patient comfort and satisfaction (Hsu et al., Effect of volume and concentration on the diffusion of botulinum exotoxin A, Arch. Dermatol. 2004, 140(11):1351-1354).

Regarding platysma bands, several studies showed that botulinum toxin treatment is an alternative, effective, non-invasive method for the treatment of platysma bands (see, e.g., Brandt, F. S. and Bellman, B., Cosmetic Use of Botulinum A Exotoxin for the Aging Neck, Dermatol. Surg. 1998, 24:1232-1234; Matarasso et al., Botulinum A Exotoxin for the Management of Platysma Bands, Plast. Reconstr. Surg. 1999, 103(2):645-652; Kane, M., Nonsurgical Treatment of Platysmal Bands with Injection of Botulinum Toxin A, Plast. Reconstr. Surg. 1999, 103:656-665; Brandt, F. S. and Boker, A., Botulinum Toxin for Rejuvenation of the Neck, Clin. Dermatology 2003, 21:515-520; Kane et al., 2010, supra; Gubanova et al., Efficacy and Safety of IncobotulinumtoxinA for the Treatment of Platysmal Bands of the Aging Neck: An Open-Label, Prospective Pilot Study, J. Drugs Dermatol. 2013, 12(12):1461-1466; Prager et al., IncobotulinumtoxinA for the Treatment of Platysmal Bands: A Single-Arm, Prospective Proof-of-Concept Clinical Study, Dermatol. Surg. 2015, 41:S88-S92; see also review of Sugrue et al., Botulinum Toxin Treatment for Mild to Moderate Platysma Bands: A Systematic Review of Efficacy, Safety, and Injection Technique, Aesthet. Surg. J. 2019, 39(2):201-206).

In the published literature, significantly varying botulinum toxin doses have been reported for the treatment of platysma bands. Total doses of between 10 U and 100 U onabotulinumtoxinA (BOTOX®) (see Brandt and Bellman, 1998, supra; Kane, 1999, supra; Brandt and Boker, 2003, supra; Tamura, 2012, supra) and up to 250 U (see Matarasso et al., 1999, supra) have been used for a successful treatment of platysma bands. Furthermore, total doses of 30 U to 60 U have been described for incobotulinumtoxin A (Xeomin®) (see Gubanova et al., 2013; supra; Prager et al., 2015, supra; see also review of Sugrue et al., supra), and a total dose of 5-20 U per band has been described for abobotulinumtoxinA (Dysport™) (Kane et al., 2010, supra). Moreover, according to the consensus recommendation on the use of botulinum toxin type A in facial aesthetics (see Carruthers et al., 2004, supra), the doses per injection point were also quite variable, resulting in total doses per band of 6 U to 40+U depending on the number of injection sites (typically three to five) and doses used.

In addition, many different toxin dilutions were used in the art. For example, Matarasso et al., 1999 (supra) used 5-10 U onabotulinumtoxin A (BOTOX®) in 0.1-0.2 mL per injection point. Prager et al., 2015 (supra) injected 2.5 U incobotulinumtoxin A in a volume of 0.0625 mL per injection point, and Gubanova et al., 2013 (supra), injected 5 U incobotulinumtoxin A (Xeomin®) in 0.1 mL per injection point. Furthermore, as disclosed in the textbook “Illustrated Guide to Aesthestic Botulinum Toxin Injections” (Kane M. and Sattler, G., 2013, Quintessence Publishing Co Ltd), the recommended dose for platysma bands is 1-2 U for incobotulinumtoxin A and onabotulinumtoxin A in 0.025 to 0.05 mL per injection point.

The aging neck is not only characterized by platysma bands but also by a loss of the mandibular definition (mandibular part of the platysma). Following treatment of platysma bands an overall tightening of the neck and a recontouring of the jawline (Brandt and Bellmann, 1998, supra; Tamura 2012, supra) or a lifting of the lower face was observed (Kane et al., 2010, supra). With special focus on the jawline, Levy et al. introduced a new technique, called the “Nefertiti lift” (see Levy, P. M., The ‘Nefertiti lift’: A new technique for specific re-contouring of the jawline, J. Cosm. Laser Ther. 2007, 9:249-252). In addition to treating platysma bands, Levy injected botulinum toxin A (Vistabel®) in the part of the platysma along and under the mandible posterior to the nasolabial fold. Injection with botulinum toxin A was found to release the tension on the5awlinee by reducing the depressor effect of the platysma and thus the elevator muscles prevail, leading to an uplift of the skin and re-contouring of the jawline.

Another injection pattern was published by de Almeida et al. (de Almeida et al., The Facial Platysma and Its Underappreciated Role in Lower Face Dynamics and Contour, Dermatol. Surg. 2017, 43:1042-1049), which not only addresses the lateral mandibular part of the platysma as described by Levy et al. (supra) but also treats the anterior fibers of the platysma and the mentalis muscle. This treatment pattern was reported to result in a redefinition of the jawline as well as an improvement of lateral cheek lines and horizontal lines along and under the mandible. Yet another technique for the improvement of mandibular contour and definition was described by Bravo et al. (Bravo et al., J. Clin. Aesthet. Dermatol. 2019, 12(11):32-34) involving a first injection point at 2-cm distance from the edge of the mandible and two further injection points anterior and superior to the first injection point.

Despite the developments of the prior art, there remains a need for an improved rejuvenation treatment of the lower face and neck by botulinum toxin that results in highly satisfying cosmetic results without compromising the safety of the patient.

### OBJECT OF THE INVENTION

It is therefore an object of the present invention to provide a botulinum toxin treatment for the rejuvenation of the lower face and neck that is highly efficient and safe.

### SUMMARY OF THE INVENTION

The present invention is based on the finding that administration of a concentrated botulinum toxin formulation at a specific dose and volume to both the platysma bands and the platysma muscle at the jawline, allows for the efficient and safe rejuvenation of the lower face and neck. The specific dosing regimen and injection scheme used in accordance with the present invention localizes the toxin to the targeted platysma bands and the platysma muscle at the jawline, thereby providing the desired cosmetic effects and at the same time preventing migration of the toxin to non-intended muscles. A small migration potential is highly important to avoid spread to vital anatomic structures in the neck, where muscles such as the deglutition muscles, the larynx and the neck flexors are in very close vicinity. The “high concentration” (or “low volume-high dose”) approach of the present invention avoids such complications while at the same time providing a high efficacy.

In a first aspect, the present invention relates to the use of botulinum toxin for the rejuvenation of the neck and lower face by injecting botulinum toxin into platysma, wherein the botulinum toxin is injected in two to four platysma bands of a subject and in the platysma muscle at a jawline on both sides of the subject's face, wherein the botulinum toxin is injected in four to five injection points per platysma band and in two to four injection points, preferably two or three injection points, and more preferably two injection points, in the platysma muscle at each jawline, and wherein the botulinum toxin is injected in the platysma bands at a dose of 3 U to 5 U and a volume of 0.04 ml to 0.06 ml per injection point and in the platysma muscle at each jawline at a dose of 3 U to 5 U and a volume of 0.04 ml to 0.06 ml per injection point.

Preferably, the dose injected in the platysma bands and the platysma muscle at the (i.e., each) jawline per injection point is 4 U to 5 U. The volume per injection point for both the platysma bands and the platysma muscle at the jawline is preferably 0.04 ml to 0.055 ml or 0.040 ml to 0.055 ml, more preferably 0.045 ml to 0.055 ml, and most preferably 0.050 ml. This means that a highly concentrated botulinum toxin solution is used within the present invention.

The high concentration (or high dose/low volume) mode of administration is particularly suitable for relatively thin muscles like the platysma muscle, not only in the neck but also in the jawline area. Botulinum toxin injections in the jawline improve the rejuvenation effect by relaxing the platysma along the jawline, thereby antagonizing the downward pull of the platysma muscle. As a result, the elevator muscles will prevail leading to an additional lifting effect and recontouring of the jawline.

In a second aspect, the present invention relates to a method for the rejuvenation of the neck and lower face by injecting botulinum toxin into platysma, wherein the botulinum toxin is injected in two to four platysma bands of a subject and in the platysma muscle at a jawline on both sides of the subject's face, wherein the botulinum toxin is injected in four to five injection points per platysma band and in two to four injection points, preferably two or three injection points, and more preferably two injection points, in the platysma muscle at each jawline, and wherein the botulinum toxin is injected in the platysma bands at a dose of 3 U to 5 U and a volume of 0.04 ml to 0.06 ml per injection point and in the platysma muscle at each jawline at a dose of 3 U to 5 U and a volume of 0.04 ml to 0.06 ml per injection point.

Preferred embodiments are set forth in the appended dependent claims and in the following detailed description taken in connection with the examples provided therein and the accompanying figures.

### BRIEF DESCRIPTION OF THE FIGURES

FIG. 1 depicts the spread of toluidine dye in porcine muscle after intramuscular injection. (A): injection of 10 μl, 20 μl or 40 μl toluidine dye at 45° injection angle; (B) injection of 10 μl, 20 μl or 40 μl toluidine dye at 90° injection angle.

FIG. 2 shows an exemplary injection pattern according to the present invention for improving platysma bands prominence and sharpness of the jawline contour (left and right side of the face with same injection pattern).

### DETAILED DESCRIPTION OF THE INVENTION

The high concentration (or high dose/low volume) approach used within the present invention allows not only for the safe administration of botulinum toxin to platysma bands and the platysma muscle at the jawline but also results in an excellent treatment efficacy with a sustained duration of the desired rejuvenation effect of the lower face and neck.

The relatively low volume is believed to prevent spreading of the toxin to adjacent muscles, thus preventing complications and unwanted side effects. This is particularly important in case of platysma because the platysma is a rather thin muscle and in anatomical vicinity to vital anatomic structures like the deglutition muscles, the larynx and the neck flexors. Special care is therefore required to prevent complications associated with the treatment of platysma, in particular of platysma bands, with botulinum toxin. The small volumes used in accordance with the present invention reduces the risk of migration/spread to unintended muscles, thereby removing the main concern with the use of botulinum toxin in aesthetic indications.

Although relatively small volumes are injected in accordance with the present invention, the doses administered per injection point are still relatively high due to the high concentration of botulinum toxin used, which improves the efficacy of the treatment. In addition, the relatively high doses will prolong the duration of effect which leads to longer treatment intervals, thereby enhancing patient satisfaction. Furthermore, both the platysma along the mandibular border and the platysma bands can be treated in one treatment session to achieve a complete relaxation of the platysma and consequently a rejuvenation of both the lower face and neck. The treatment of the platysma in the jawline area not only results in rejuvenation of the lower face but is also assumed to diminish residual banding and development of compensatory bands.

Overall, the present invention allows for the treatment of the platysma as an anatomic and cosmetic entity and leads to a balanced and harmonious appearance of the lower face and neck, enhancing patient satisfaction without increasing side effects. Furthermore, it enables the use of longer treatment intervals, resulting in a lower immunogenic risk of neutralizing antibody formation which is known to lead to secondary treatment failure. As a result, patient convenience and satisfaction are improved. Thus, the botulinum toxin treatment at low volumes and high doses (i.e., using botulinum toxin at high concentrations) provides high efficacy, excellent aesthetic outcome and long duration of effect, while preventing or minimizing unwanted side effects.

In a first aspect, the present invention relates to the use of botulinum toxin for the rejuvenation of the neck and lower face by injecting botulinum toxin into platysma, wherein the botulinum toxin is injected in two to four platysma bands of a subject and in the platysma muscle at a jawline on both sides of the subject's face, wherein the botulinum toxin is injected in four to five injection points per platysma band and in two to four (e.g., 2, 3 or 4) injection points in the platysma muscle at each jawline, and wherein the botulinum toxin is injected in the platysma bands at a dose of 3 U to 5 U and a volume of 0.04 ml to 0.06 ml per injection point and in the platysma muscle at each jawline at a dose of 3 U to 5 U and a volume of 0.04 ml to 0.06 ml per injection point.

As used herein, the term “rejuvenation” broadly means restoring a youthful appearance. Further, the term “rejuvenation” when used in connection with the use and method of the present invention refers to a purely aesthetic application. This is, in accordance with the present invention, botulinum toxin is only used for cosmetic purposes.

The term “lower face”, as used herein, refers to the area from the subnasale to the menton, comprising jawline including jowls and marionette lines. The term “neck”, as used herein, refers to the region extending from the lower part of the mandible (lower jawbone) down to the clavicle and sternum.

As used herein, the term “platysma” has the same meaning as the term “platysma muscle” and these terms are therefore interchangeably used herein. The platysma is a thin sheet of muscle that envelopes the entire neck. It arises from the fascia that covers the upper segments of the deltoid and pectoralis muscles and a portion of the platysma muscle extends over the mandibular border and inserts into the lower face just above the mandible. Thus, since only the platysma is targeted in this invention, injections into the jawline should be understood to refer to injections into the platysma muscle at the jawline. Since this part of the platysma muscle inserts on the lower face, these injections are intended to result in a rejuvenation of the lower face.

The term “platysma bands”, as used herein, has the meaning known in the art, and refers to cords or bands of the neck. The platysma bands are covered by the term “platysma” or the term “platysma muscle” as used herein. Platysma bands are characteristic of an ageing neck. In the ageing process the platysma muscle begins to separate and bunch into cords or bands, usually in the midline para median at each side of the neck but also at the side of the neck. Hence, platysma bands may be, and usually are, medial and lateral, with two medial and two lateral bands. In the context of the present invention, the term “injection in platysma bands”, is intended to refer to injection into the platysma muscle, i.e., intramuscular injection. The injection of botulinum toxin into platysma bands can relax and flatten the platysma bands to make the bands less pronounced. It should be noted that the term “platysma bands”, as used herein, refers to vertical bands, i.e., bands than run vertically across the front of the neck. Thus, “platysma bands” are different to necklace lines, i.e., horizontal bands or lines that run horizontally around front of the neck.

The term “jawline”, as used herein, refers to the lower edge of the side of the face below the cheek, defined by the mandible. The singular use of “jawline” is intended to refer to the jawlines on both sides (left and right sides) of the face, unless specifically stated otherwise or the context indicates otherwise. Further, as mentioned above, the platysma crosses the mandible, with some fibers being inserted into the bone below the oblique line and others extending approximately 4 cm above the mandibular border into the cheek and inserting into the skin and subcutaneous tissue. Therefore, as used herein, the term “injected in the jawlines” or “injection at the jawline” or similar terms are intended to mean that the platysma muscle is injected and, more specifically, are intended to refer to an intramuscular injection in the part of the platysma along or under the mandible posterior to the nasolabial fold. Without being bound by theory, it is believed that injections in the jawline leads to a release of the tension on the jawline by reducing the depressor effect of the platysma. As a result, the elevator muscles prevail, leading to an uplift of the skin and re-contouring of the jawline.

The term “subject”, as used herein, is not particularly limited and refers to a human, who is in the need of cosmetic treatment of the neck, the lower face, or the neck and lower face. The term “subject” may be interchangeably used with the term patient in the context of the present invention. The subject has preferably an age of at least 30 years, more preferably at least 40 years or at least 50 years, and most preferably at least 60 years.

Within the meaning of the present invention, numerical values without decimal places shall be understood to include all numerical values with one or more decimal places that, applying common rules of rounding, give the numerical value without decimal places. For example, the numerical value 3 includes 2.5 or 2.50 (which are rounded up to 3) and 3.4 or 3.49 (which are rounded off to 3), and all numerical values in between. As regards endpoints of ranges, the same applies, i.e., a range of 3 to 5 may mean, e.g., 2.5-3.4 to 4.5-5.4. It is also contemplated that each numerical value or one or both, preferably both, endpoints of ranges disclosed in this application, if they have no decimal places, can be a numerical value with a decimal place that is zero, e.g., 4 may be 4.0.

The term “botulinum toxin”, as used herein, is not particularly limited and includes botulinum toxin of any serotype (e.g., BoNT/A-H. For example, the botulinum toxin may be of serotype A or B (BoNT/A, BONT/B). Preferably, the botulinum toxin is of serotype A, more preferably of serotype A1 (BoNT/A1), and most preferably BoNT/A1 produced by Clostridium botulinum Hall strain. Furthermore, as used herein, the term “botulinum toxin” (“BT”) and the synonymously used term “botulinum neurotoxin” (“BONT”) are intended to refer to the pure botulinum neurotoxin and/or any complex thereof, i.e., any complex of the pure botulinum neurotoxin and complexing proteins (referred to as the “toxin complex”). Preferably, the botulinum toxin is the pure botulinum neurotoxin of serotype A.

The term “pure botulinum neurotoxin”, as used herein, means the botulinum neurotoxin free of complexing proteins (sometimes also referred to as the “neurotoxic component”), or more precisely, the botulinum neurotoxin without neurotoxin-associated complexing proteins (NAPs). The pure botulinum neurotoxin is the (active) neurotoxic polypeptide that ultimately inhibits acetylcholine release. It is a di-chain protein comprised of a light chain (LC; about 50 kDa) and a heavy chain (HC; about 100 kDa), held together by a disulfide bond. The active neurotoxic polypeptide may therefore also be referred to herein as the “150 kDa neurotoxin”, “Clostridium botulinum neurotoxin (150 kD)” or “neurotoxic component”. Preferably, the botulinum toxin is the (pure) botulinum neurotoxin contained in Xeomin® or is Xeomin®.

The term “toxin complex”, as used herein, refers to a high-molecular complex of the neurotoxic component and a set of complexing proteins (NAPs), such as the 900 kDa, 500 kDa, and 300 kDa C. botulinum type A toxin complexes. The complexing proteins are nontoxic nonhaemagglutinin (NTNHA) and, in strains of serotype A-D, different haemagglutinins (HAs). For example, the 900 kDa complex is included in onabotulinumtoxin A (Botox®/Vistabel®, Allergan, Inc., Irvine, CA, USA), and abobotulinumtoxin A (Dysport®, Azzalure®, Ipsen, Paris, France), Alluzience® (Ipsen/Galderma) and Innotox® (Medytox) also contain a toxin complex as active agent. Preferably, the botulinum toxin is, besides the pure botulinum neurotoxin that is contained in Xeomin® or is Xeomin®, the toxin complex contained in Botox® or Dysport®, or is Botox® or Dysport®. In another preferred embodiment, the botulinum toxin is not the toxin complex contained in Dysport® or is not Dysport®.

The botulinum toxin may be a natural neurotoxin obtainable from Clostridium botulinum or any other botulinum toxin such as a botulinum toxin obtainable from alternative sources, including recombinant technologies and genetic or chemical modification. Chimeric or genetically modified botulinum toxins, i.e., botulinum toxins containing mutations including substitutions, deletions and insertions, are also encompassed by the terms “botulinum toxin”, “neurotoxic component” and the like. Preferably, the mutation does not compromise any of the biological activities of botulinum toxin. However, it is also envisaged to use mutations to modulate the biological activity of the botulinum toxin. Also included are botulinum toxins containing chemically modified amino acids, for example one or more amino acids which are glycosylated, acetylated or otherwise modified. This may be beneficial to the uptake or stability of the toxin. Particularly preferred is the lipidation of the neurotoxic component.

Within the present invention, the dose is expressed in biological units because the used botulinum toxin may contain, for example, variable percentages of inactive toxin that contribute to the overall protein load without contributing to efficacy. Within the context of the present invention, the biological potency of botulinum toxin is determined using the mouse bioassay (MBA). The MBA determines the median lethal dose (LD₅₀) of toxin/neurotoxin after intraperitoneal injection in mice, i.e., the dose of toxin/neurotoxin capable of killing 50% of a group of mice. On this basis, 1 unit (U) of toxin/neurotoxin, as used herein, is defined as one mouse LD50 (1.0 LD50=1.0 U). The LD50 mouse bioassay is the gold standard among various biological, chemical or immunological detection methods for botulinum toxin and is known to those skilled in the art (see, e.g., Pearce, L. B.; Borodic, G. E.; First, E. R.; MacCallum, R. D. Measurement of botulinum toxin activity: Evaluation of the lethality assay. Toxicol. Appl. Pharmacol. 1994, 128, 69-77).

Another useful method for determining the biological activity (biological potency) of a botulinum neurotoxin is a cell-based potency assay which is disclosed, for example, in WO2009/114748, WO 2013/049508 or WO 2014/207109. The activity results obtained with such cell-based assays correspond to the activity values obtained in the mouse i.p. LD50 assay because the values are calibrated using the LD50 reference standard.

Due to differences in the LD50 tests used by manufacturers of commercial botulinum toxin formulations, the unit potencies indicated by the manufacturers for their commercial botulinum toxin formulations is proprietary and cannot easily be compared. Therefore, within the framework of the present invention, the conversion rates provided below are used to establish the comparative potencies of incobotulinumtoxinA (“INCO”; Xeomin®, Bocouture®; botulinum toxin serotype A, free of complexing proteins; Merz Pharmaceuticals GmbH), onabotulinumtoxinA (“ONA”; Botox®, Vistabel®; botulinum toxin complex of serotype A; Allergan Inc.), abobotulinumtoxinA (“ABO”; Dysport®, Azzalure®; botulinum toxin complex of serotype A; Medicis Pharmaceutical Corp., Galderma Lab.), rimabotulinumtoxinB (“RIM”; Myobloc®), NeuroBloc®; botulinum toxin serotype B; Solstice Neurosciences Inc.), and PurTox® (“TBD”; botulinum toxin serotype A; Mentor Worldwide LLC). For use herein, the conversion rate of ONA and INCO is 1:1. The conversion rate of ONA/INCO: ABO is 1:2.5. The conversion rate of ONA/INCO:RIM is 1:50, and the conversion rate of ONA/INCO:TBD is 1:1.5. Furthermore, and preferably, within the context of the present invention, 1 U of INCO (Xeomin®) and 1 U of onabotulinumtoxinA (“ONA”; Botox®) shall be deemed to correspond to one mouse LD50 (1.0 LD50), or 1 U, measured using the MBA as described above. It is further preferred within the present invention that, if the botulinum toxin is abobotulinumtoxinA, 2.5 U of abobotulinumtoxinA equal 1 U, with 1 U being preferably one mouse LD50 (1.0 LD50), measured using the above-described MBA.

In a preferred embodiment, the first aspect of the present invention relates to the use of botulinum toxin for the rejuvenation of the neck and lower face by injecting botulinum toxin into platysma, wherein the botulinum toxin is injected in two to four platysma bands of a subject and in the platysma muscle at a jawline on both sides of the subject's face, wherein the botulinum toxin is injected in four to five injection points per platysma band and in two to four injection points in the platysma muscle at each jawline, and wherein the botulinum toxin is injected in the platysma bands at a dose of 3 U to 5 U and a volume of 0.04 ml to 0.06 ml per injection point and in the platysma muscle at each jawline at a dose of 3 U to 5 U and a volume of 0.04 ml to 0.06 ml per injection point, with the proviso that, if the botulinum toxin is abobotulinumtoxinA, 2.5 U of abobotulinumtoxinA equal 1 U. In another preferred embodiment, the first aspect of the present invention relates to the use of botulinum toxin for the rejuvenation of the neck and lower face by injecting botulinum toxin into platysma, wherein the botulinum toxin is injected in two to four platysma bands of a subject and in the platysma muscle at a jawline on both sides of the subject's face, wherein the botulinum toxin is injected in four to five injection points per platysma band and in two to four injection points in the platysma muscle at each jawline, and wherein the botulinum toxin is injected in the platysma bands at a dose of 3 U to 5 U and a volume of 0.04 ml to 0.06 ml per injection point and in the platysma muscle at each jawline at a dose of 3 U to 5 U and a volume of 0.04 ml to 0.06 ml per injection point, wherein 1 U corresponds to 1 LD50, with LD50 being the median lethal dose as measured using the mouse LD50 bioassay, with the proviso that, if the botulinum toxin is abobotulinumtoxinA, 2.5 U of abobotulinumtoxinA equal 1 U. In yet another preferred embodiment, the first aspect of the present invention relates to the use of botulinum toxin for the rejuvenation of the neck and lower face by injecting botulinum toxin into platysma, wherein the botulinum toxin is injected in two to four platysma bands of a subject and in the platysma muscle at a jawline on both sides of the subject's face, wherein the botulinum toxin is injected in four to five injection points per platysma band and in two to four injection points in the platysma muscle at each jawline, and wherein the botulinum toxin is injected in the platysma bands at a dose of 3 U to 5 U and a volume of 0.04 ml to 0.06 ml per injection point and in the platysma muscle at each jawline at a dose of 3 U to 5 U and a volume of 0.04 ml to 0.06 ml per injection point, wherein 1 U corresponds to 1 LD50, with LD50 being the median lethal dose as measured using the mouse LD50 bioassay, with the proviso that, if the botulinum toxin is incobotulinumtoxinA or onabotulinumtoxinA, 1 U of incobotulinumtoxinA and 1 U of onabotulinumtoxinA both equal 1 U, and, if the botulinumtoxin is abobotulinumtoxinA, 2.5 U of abobotulinumtoxinA equal 1 U.

As used herein, the term “comprising”, like the terms “including” and containing”, and any variations thereof such as “comprises”, “includes” and “contains”, are intended to refer to a non-exclusive inclusion, such that a process, method, product-by-process, composition or formulation that comprises, includes, or contains an element or list of elements does not include only those elements but can include other elements not expressly listed for such process, method, product-by-process, composition or formulation. In addition, within the framework of the present invention, it is intended that each of the terms “comprise,” “comprising”, “includes”, “including”, “contains”, “containing”, and any variations thereof, can be replaced by the term “consists” or “consisting”, or any variation thereof, which will be understood to refer to an exclusive inclusion of the elements indicated.

The terms “a” and “an” and “the” and similar reference used in the context of the present invention are to be construed to cover both the singular and the plural and, thus, may also relate to “at least one” or “more than one”, unless otherwise indicated herein or clearly contradicted by the context.

In accordance with the first aspect of the present invention, the botulinum toxin is injected in the platysma bands at a dose of 3 U to 5 U and a volume of 0.04 ml to 0.06 ml per injection point and (independently thereof) in the platysma muscle at each jawline (i.e., the jawline on the left and right sides of the subject's face) at a dose of 3 U to 5 U and a volume of 0.04 ml to 0.06 ml per injection point.

The botulinum toxin is preferably injected in the platysma bands at a dose of between 3.0 U and 5.0 U (e.g., 3.1 U, 3.5 U, 4.0 U, 4.5 U, and 4.9 U) per injection point, more preferably at a dose of between 4 U and 5 U, in particular between 4.0 U and 5.0 U (e.g., 4.1 U, 4.5 U, and 4.9 U) per injection point. Further preferably, the botulinum toxin is injected in the platysma bands at a volume of 0.045 ml to 0.06 ml (preferably 0.045 ml to 0.060 ml) or 0.04 ml to 0.055 ml (preferably 0.040 ml to 0.055 ml) per injection point, more preferably at a volume of 0.045 ml to 0.055 ml per injection point, and most preferably at a volume of 0.05 ml per injection point.

The botulinum toxin is preferably injected in the platysma muscle at the jawline on the left and right (i.e., both) sides of the subject's face at a dose of between 3.0 U and 5.0 U (e.g., 3.1 U, 3.5 U, 4.0 U, 4.5 U, and 4.9 U) per injection point, more preferably at a dose of between 4 U and 5 U, in particular between 4.0 U and 5.0 U (e.g., 4.1 U, 4.5 U, and 4.9 U) per injection point. Further preferably, the botulinum toxin is injected in the platysma muscle at both jawlines at a volume of 0.045 ml to 0.06 ml (preferably 0.045 ml to 0.060 ml) or 0.04 ml to 0.055 ml (preferably 0.040 ml to 0.055 ml) per injection point, more preferably at a volume of 0.045 ml to 0.055 ml per injection point, and most preferably at a volume of 0.05 ml per injection point.

It is contemplated within the present invention that the dose and volume administered per injection point in the platysma bands may be identical or different from point to point, provided the dose and volume per injection point is within the range indicated (e.g., from 3 U to 5 U and from 0.04 ml to 0.06 ml). Likewise, the dose and volume administered per injection point in the platysma muscle at the jawline on both sides of the subject's face may be identical or different from point to point, provided the dose or volume per injection point is within the range indicated (e.g., from 3 U to 5 U and from 0.04 ml to 0.06 ml). Also, in accordance with the present invention, the doses and volumes injected in the platysma bands are independently selected from the doses and volumes injected in both the left and right jawline.

Within the present invention, the dose and volume per injection point in the platysma bands are preferably identical for all injection points. Likewise, it is preferred that the dose and volume per injection point in the platysma muscle at the jawline on both sides of the patient's face are identical for all injection points. More preferably, the dose and volume administered to each injection point in both the platysma bands and the platysma muscle at the jawline (on the left and right side of the patient's face) are identical.

In one preferred embodiment, the botulinum toxin is injected in the platysma bands at a dose of 3 U to 5 U (preferably 3.0 U to 5.0 U) and a volume of 0.04 ml to 0.06 ml (preferably 0.040 ml to 0.060 ml) per injection point and in the platysma muscle at each jawline at a dose of 3 U to 5 U (preferably 3.0 U to 5.0 U) and a volume of 0.04 ml to 0.06 ml (preferably 0.040 ml to 0.060 ml) per injection point. In one preferred embodiment, the botulinum toxin is injected in the platysma bands at a dose of 3 U to 4 U (preferably 3.0 U to 4.0 U) and a volume of 0.04 ml to 0.06 ml (preferably 0.040 ml to 0.060 ml) per injection point and in the platysma muscle at each jawline at a dose of 3 U to 5 U (preferably 3.0 U to 5.0 U) and a volume of 0.04 ml to 0.06 ml (preferably 0.040 ml to 0.060 ml) per injection point. In one preferred embodiment, the botulinum toxin is injected in the platysma bands at a dose of 4 U to 5 U (preferably 4.0 U to 5.0 U) and a volume of 0.04 ml to 0.06 ml (preferably 0.040 ml to 0.060 ml) per injection point and in the platysma muscle at each jawline at a dose of 3 U to 5 U (preferably 3.0 U to 5.0 U) and a volume of 0.04 ml to 0.06 ml (preferably 0.040 ml to 0.060 ml) per injection point. In one preferred embodiment, the botulinum toxin is injected in the platysma bands at a dose of 3 U to 5 U (preferably 3.0 U to 5.0 U) and a volume of 0.04 ml to 0.06 ml (preferably 0.040 ml to 0.060 ml) per injection point and in the platysma muscle at each jawline at a dose of 3 U to 4 U (preferably 3.0 U to 4.0 U) and a volume of 0.04 ml to 0.06 ml (preferably 0.040 ml to 0.060 ml) per injection point. In one preferred embodiment, the botulinum toxin is injected in the platysma bands at a dose of 3 U to 4 U (preferably 3.0 U to 4.0 U) and a volume of 0.04 ml to 0.06 ml (preferably 0.040 ml to 0.060 ml) per injection point and in the platysma muscle at each jawline at a dose of 3 U to 4 U (preferably 3.0 U to 4.0 U) and a volume of 0.04 ml to 0.06 ml (preferably 0.040 ml to 0.060 ml) per injection point. In one preferred embodiment, the botulinum toxin is injected in the platysma bands at a dose of 4 U to 5 U (preferably 4.0 U to 5.0 U) and a volume of 0.04 ml to 0.06 ml (preferably 0.040 ml to 0.060 ml) per injection point and in the platysma muscle at each jawline at a dose of 3 U to 4 U (preferably 3.0 U to 4.0 U) and a volume of 0.04 ml to 0.06 ml (preferably 0.040 ml to 0.060 ml) per injection point. In one preferred embodiment, the botulinum toxin is injected in the platysma bands at a dose of 3 U to 5 U (preferably 3.0 U to 5.0 U) and a volume of 0.04 ml to 0.06 ml (preferably 0.040 ml to 0.060 ml) per injection point and in the platysma muscle at each jawline at a dose of 4 U to 5 U (preferably 4.0 U to 5.0 U) and a volume of 0.04 ml to 0.06 ml (preferably 0.040 ml to 0.060 ml) per injection point. In one preferred embodiment, the botulinum toxin is injected in the platysma bands at a dose of 3 U to 4 U (preferably 3.0 U to 4.0 U) and a volume of 0.04 ml to 0.06 ml (preferably 0.040 ml to 0.060 ml) per injection point and in the platysma muscle at each jawline at a dose of 4 U to 5 U (preferably 4.0 U to 5.0 U) and a volume of 0.04 ml to 0.06 ml (preferably 0.040 ml to 0.060 ml) per injection point. In one preferred embodiment, the botulinum toxin is injected in the platysma bands at a dose of 4 U to 5 U (preferably 4.0 U to 5.0 U) and a volume of 0.04 ml to 0.06 ml (preferably 0.040 ml to 0.060 ml) per injection point and in the platysma muscle at each jawline at a dose of 4 U to 5 U (preferably 4.0 U to 5.0 U) and a volume of 0.04 ml to 0.06 ml (preferably 0.040 ml to 0.060 ml) per injection point.

In another preferred embodiment, the botulinum toxin is injected in the platysma bands at a dose of 3 U to 5 U (preferably 3.0 U to 5.0 U) and a volume of 0.04 ml to 0.055 ml (preferably 0.040 ml to 0.055 ml) per injection point and in the platysma muscle at each jawline at a dose of 3 U to 5 U (preferably 3.0 U to 5.0 U) and a volume of 0.04 ml to 0.055 ml (preferably 0.040 ml to 0.055 ml) per injection point. In another preferred embodiment, the botulinum toxin is injected in the platysma bands at a dose of 3 U to 4 U (preferably 3.0 U to 4.0 U) and a volume of 0.04 ml to 0.055 ml (preferably 0.040 ml to 0.055 ml) per injection point and in the platysma muscle at each jawline at a dose of 3 U to 5 U (preferably 3.0 U to 5.0 U) and a volume of 0.04 ml to 0.055 ml (preferably 0.040 ml to 0.055 ml) per injection point. In another preferred embodiment, the botulinum toxin is injected in the platysma bands at a dose of 4 U to 5 U (preferably 4.0 U to 5.0 U) and a volume of 0.04 ml to 0.055 ml (preferably 0.040 ml to 0.055 ml) per injection point and in the platysma muscle at each jawline at a dose of 3 U to 5 U (preferably 3.0 U to 5.0 U) and a volume of 0.04 ml to 0.055 ml (preferably 0.040 ml to 0.055 ml) per injection point. In another preferred embodiment, the botulinum toxin is injected in the platysma bands at a dose of 3 U to 5 U (preferably 3.0 U to 5.0 U) and a volume of 0.04 ml to 0.055 ml (preferably 0.040 ml to 0.055 ml) per injection point and in the platysma muscle at each jawline at a dose of 3 U to 4 U (preferably 3.0 U to 4.0 U) and a volume of 0.04 ml to 0.055 ml (preferably 0.040 ml to 0.055 ml) per injection point. In another preferred embodiment, the botulinum toxin is injected in the platysma bands at a dose of 3 U to 4 U (preferably 3.0 U to 4.0 U) and a volume of 0.04 ml to 0.055 ml (preferably 0.040 ml to 0.055 ml) per injection point and in the platysma muscle at each jawline at a dose of 3 U to 4 U (preferably 3.0 U to 4.0 U) and a volume of 0.04 ml to 0.055 ml (preferably 0.040 ml to 0.055 ml) per injection point. In another preferred embodiment, the botulinum toxin is injected in the platysma bands at a dose of 4 U to 5 U (preferably 4.0 U to 5.0 U) and a volume of 0.04 ml to 0.055 ml (preferably 0.040 ml to 0.055 ml) per injection point and in the platysma muscle at each jawline at a dose of 3 U to 4 U (preferably 3.0 U to 4.0 U) and a volume of 0.04 ml to 0.055 ml (preferably 0.040 ml to 0.055 ml) per injection point. In another preferred embodiment, the botulinum toxin is injected in the platysma bands at a dose of 3 U to 5 U (preferably 3.0 U to 5.0 U) and a volume of 0.04 ml to 0.055 ml (preferably 0.040 ml to 0.055 ml) per injection point and in the platysma muscle at each jawline at a dose of 4 U to 5 U (preferably 4.0 U to 5.0 U) and a volume of 0.04 ml to 0.055 ml (preferably 0.040 ml to 0.055 ml) per injection point. In another preferred embodiment, the botulinum toxin is injected in the platysma bands at a dose of 3 U to 4 U (preferably 3.0 U to 4.0 U) and a volume of 0.04 ml to 0.055 ml (preferably 0.040 ml to 0.055 ml) per injection point and in the platysma muscle at each jawline at a dose of 4 U to 5 U (preferably 4.0 U to 5.0 U) and a volume of 0.04 ml to 0.055 ml (preferably 0.040 ml to 0.055 ml) per injection point. In another preferred embodiment, the botulinum toxin is injected in the platysma bands at a dose of 4 U to 5 U (preferably 4.0 U to 5.0 U) and a volume of 0.04 ml to 0.055 ml (preferably 0.040 ml to 0.055 ml) per injection point and in the platysma muscle at each jawline at a dose of 4 U to 5 U (preferably 4.0 U to 5.0 U) and a volume of 0.04 ml to 0.055 ml (preferably 0.040 ml to 0.055 ml) per injection point.

In yet another preferred embodiment, the botulinum toxin is injected in the platysma bands at a dose of 3 U to 5 U (preferably 3.0 U to 5.0 U) and a volume of 0.045 ml to 0.055 ml (most preferably 0.050 ml) per injection point and in the platysma muscle at each jawline at a dose of 3 U to 5 U (preferably 3.0 U to 5.0 U) and a volume of 0.045 ml to 0.055 ml (most preferably 0.050 ml) per injection point. In yet another preferred embodiment, the botulinum toxin is injected in the platysma bands at a dose of 3 U to 4 U (preferably 3.0 U to 4.0 U) and a volume of 0.045 ml to 0.055 ml (most preferably 0.050 ml) per injection point and in the platysma muscle at each jawline at a dose of 3 U to 5 U (preferably 3.0 U to 5.0 U) and a volume of 0.045 ml to 0.055 ml (most preferably 0.050 ml) per injection point. In yet another preferred embodiment, the botulinum toxin is injected in the platysma bands at a dose of 4 U to 5 U (preferably 4.0 U to 5.0 U) and a volume of 0.045 ml to 0.055 ml (most preferably 0.050 ml) per injection point and in the platysma muscle at each jawline at a dose of 3 U to 5 U (preferably 3.0 U to 5.0 U) and a volume of 0.045 ml to 0.055 ml (most preferably 0.050 ml) per injection point. In yet another preferred embodiment, the botulinum toxin is injected in the platysma bands at a dose of 3 U to 5 U (preferably 3.0 U to 5.0 U) and a volume of 0.045 ml to 0.055 ml (most preferably 0.050 ml) per injection point and in the platysma muscle at each jawline at a dose of 3 U to 4 U (preferably 3.0 U to 4.0 U) and a volume of 0.045 ml to 0.055 ml (most preferably 0.050 ml) per injection point. In yet another preferred embodiment, the botulinum toxin is injected in the platysma bands at a dose of 3 U to 4 U (preferably 3.0 U to 4.0 U) and a volume of 0.045 ml to 0.055 ml (most preferably 0.050 ml) per injection point and in the platysma muscle at each jawline at a dose of 3 U to 4 U (preferably 3.0 U to 4.0 U) and a volume of 0.045 ml to 0.055 ml (most preferably 0.050 ml) per injection point. In yet another preferred embodiment, the botulinum toxin is injected in the platysma bands at a dose of 4 U to 5 U (preferably 4.0 U to 5.0 U) and a volume of 0.045 ml to 0.055 ml (most preferably 0.050 ml) per injection point and in the platysma muscle at each jawline at a dose of 3 U to 4 U (preferably 3.0 U to 4.0 U) and a volume of 0.045 ml to 0.055 ml (most preferably 0.050 ml) per injection point. In yet another preferred embodiment, the botulinum toxin is injected in the platysma bands at a dose of 3 U to 5 U (preferably 3.0 U to 5.0 U) and a volume of 0.045 ml to 0.055 ml (most preferably 0.050 ml) per injection point and in the platysma muscle at each jawline at a dose of 4 U to 5 U (preferably 4.0 U to 5.0 U) and a volume of 0.045 ml to 0.055 ml (most preferably 0.050 ml) per injection point. In yet another preferred embodiment, the botulinum toxin is injected in the platysma bands at a dose of 3 U to 4 U (preferably 3.0 U to 4.0 U) and a volume of 0.045 ml to 0.055 ml (most preferably 0.050 ml) per injection point and in the platysma muscle at each jawline at a dose of 4 U to 5 U (preferably 4.0 U to 5.0 U) and a volume of 0.045 ml to 0.055 ml (most preferably 0.050 ml) per injection point. In yet another preferred embodiment, the botulinum toxin is injected in the platysma bands at a dose of 4 U to 5 U (preferably 4.0 U to 5.0 U) and a volume of 0.045 ml to 0.055 ml (most preferably 0.050 ml) per injection point and in the platysma muscle at each jawline at a dose of 4 U to 5 U (preferably 4.0 U to 5.0 U) and a volume of 0.045 ml to 0.055 ml (most preferably 0.050 ml) per injection point.

In still another preferred embodiment, the botulinum toxin is injected in the platysma bands at a dose of 3 U and a volume of 0.045 ml to 0.055 ml (preferably 0.050 ml) per injection point and in the platysma muscle at each jawline at a dose of 3 U and a volume of 0.045 ml to 0.055 ml (preferably 0.050 ml) per injection point. In still another preferred embodiment, the botulinum toxin is injected in the platysma bands at a dose of 3 U and a volume of 0.045 ml to 0.055 ml (preferably 0.050 ml) per injection point and in the platysma muscle at each jawline at a dose of 4 U and a volume of 0.045 ml to 0.055 ml (preferably 0.050 ml) per injection point. In still another preferred embodiment, the botulinum toxin is injected in the platysma bands at a dose of 3 U and a volume of 0.045 ml to 0.055 ml (preferably 0.050 ml) per injection point and in the platysma muscle at each jawline at a dose of 5 U and a volume of 0.045 ml to 0.055 ml (preferably 0.050 ml) per injection point. In still another preferred embodiment, the botulinum toxin is injected in the platysma bands at a dose of 4 U and a volume of 0.045 ml to 0.055 ml (preferably 0.050 ml) per injection point and in the platysma muscle at each jawline at a dose of 3 U and a volume of 0.045 ml to 0.055 ml (preferably 0.050 ml) per injection point. In still another preferred embodiment, the botulinum toxin is injected in the platysma bands at a dose of 4 U and a volume of 0.045 ml to 0.055 ml (preferably 0.050 ml) per injection point and in the platysma muscle at each jawline at a dose of 4 U and a volume of 0.045 ml to 0.055 ml (preferably 0.050 ml) per injection point. In still another preferred embodiment, the botulinum toxin is injected in the platysma bands at a dose of 4 U and a volume of 0.045 ml to 0.055 ml (preferably 0.050 ml) per injection point and in the platysma muscle at each jawline at a dose of 5 U and a volume of 0.045 ml to 0.055 ml (preferably 0.050 ml) per injection point. In still another preferred embodiment, the botulinum toxin is injected in the platysma bands at a dose of 5 U and a volume of 0.045 ml to 0.055 ml (preferably 0.050 ml) per injection point and in the platysma muscle at each jawline at a dose of 3 U and a volume of 0.045 ml to 0.055 ml (preferably 0.050 ml) per injection point. In still another preferred embodiment, the botulinum toxin is injected in the platysma bands at a dose of 5 U and a volume of 0.045 ml to 0.055 ml (preferably 0.050 ml) per injection point and in the platysma muscle at each jawline at a dose of 4 U and a volume of 0.045 ml to 0.055 ml (preferably 0.050 ml) per injection point. In still another preferred embodiment, the botulinum toxin is injected in the platysma bands at a dose of 5 U and a volume of 0.045 ml to 0.055 ml (preferably 0.050 ml) per injection point and in the platysma muscle at each jawline at a dose of 5 U and a volume of 0.045 ml to 0.055 ml (preferably 0.050 ml) per injection point.

Moreover, in accordance with the present invention, the botulinum toxin is preferably injected at a concentration of 50 U/ml to 125 U/ml, more preferably 60 U/ml to 120 U/ml, still more preferably 70 U/ml to 110 U/ml, and most preferably 80 U/ml to 100 U/ml. In particular, in the above-described “one preferred embodiment”, “another preferred embodiment” and “yet another preferred embodiment”, the botulinum toxin is preferably injected at a concentration of 50 U/ml to 125 U/ml, more preferably 60 U/ml to 120 U/ml, still more preferably 70 U/ml to 110 U/ml, and most preferably 80 U/ml to 100 U/ml. An appropriate concentration can be readily selected by the skilled person depending on the dose to be injected and taking into account that the injection volume should not be too low for practicability reasons and to avoid dosing inaccuracies.

With respect to the injection scheme, the botulinum toxin is injected in two to four platysma bands. Preferably, the botulinum toxin is injected in the two left and right medial bands or the two left and right lateral bands. More preferably, the botulinum toxin is injected in four platysma bands of a subject, namely each of the left and right medial and lateral bands.

Furthermore, within the context of the present invention, the botulinum toxin is injected in four or five injection points per platysma band. Preferably, the botulinum toxin is injected in five injection points per platysma band. More preferably, the botulinum toxin is injected in two or four, preferably four, platysma bands and in four or five injection points per platysma band. Most preferably, the botulinum toxin is injected in four platysma bands and in five injection points per platysma band.

Moreover, the botulinum toxin is injected in two to four injection points in the platysma muscle at each jawline, preferably three injection points in the platysma muscle at each jawline, and more preferably two injection points in the platysma muscle at each jawline.

In accordance with the present invention, it is particularly preferred that the botulinum toxin is injected in four platysma bands of a subject and in the platysma muscle at the jawline on both sides of the subject's face, wherein the botulinum toxin is injected in two injection points in the platysma muscle at each jawline and in five injection points per platysma band. It is further particularly preferred that the botulinum toxin is injected in four platysma bands of a subject and in the platysma muscle at the jawline on both sides of the subject's face, wherein the botulinum toxin is injected in two injection points in the platysma muscle at each jawline and in five injection points per platysma band, wherein the botulinum toxin is injected at a dose of 3 U to 5 U (preferably 3.0 to 5.0 U) and a volume of 0.04 ml to 0.06 ml (preferably 0.04 ml to 0.06 ml) per injection point or wherein the botulinum toxin is injected using any of the above-described dosing regimens.

The injection points along the platysma bands are preferably spaced at a distance of about 0.5 cm to about 2.0 cm, preferably about 0.5 cm to about 1.5 cm, more preferably about 1.0 cm. This spacing refers to the distance between adjacent injection points on the platysma bands.

The injection points along each jawline are preferably spaced at a distance of about 0.5 cm to about 3.0 cm or about 1.0 cm to about 3.0 cm, more preferably at a distance of about 1.0 cm to about 2.5 cm, still more preferably at a distance of about 1.5 cm to about 2.5 cm, and most preferably at a distance of about 2.0 cm. Preferably, botulinum toxin is administered in two to four, preferably three and most preferably two, injection points per jawline on each side of the patient's face, more specifically generally per side along and under each mandible not anterior to a line drawn from the nasolabial fold down to the mandible.

According to a preferred embodiment of the present invention, the injection pattern includes five injection points in each of four platysma bands (20 injections in total) and two injection points per jawline on each side of the patient's face (4 injections in total), wherein the injection points along each platysma band are spaced at a distance of about 0.5 cm to about 2.0 cm, preferably about 0.5 cm to about 1.5 cm, more preferably about 1.0 cm and the injection points along each jawline are spaced at a distance of about 1.5 cm to about 3.0 cm, preferably about 1.5 cm to about 2.5 cm, and more preferably about 2.0 cm, or wherein the injection points along each platysma band are spaced at a distance of about 0.5 cm to about 1.5 cm and the injection points along each jawline are spaced at a distance of about 1.5 cm to about 2.5 cm.

In accordance with the use according to the first aspect of the present invention, the botulinum toxin may be administered in consecutive treatment cycles. It is understood that a treatment cycle is the time interval between two administrations of the botulinum neurotoxin, i.e., a treatment cycle consists of one administration of the botulinum neurotoxin and a follow-up period until the next botulinum neurotoxin injection is administered. The said administration of botulinum toxin occurs in a treatment session (or “injection session”). The time interval between two consecutive administrations of the botulinum neurotoxin can vary between about 5 and 10 months, in particular between about 6 and 9 months or between about 7 and 8 months This prolonged treatment interval is enabled by the longer duration of effect achieved by the present invention.

The prolonged duration of effect allows for less frequent administration of botulinum toxin. For example, botulinum neurotoxin may be repeatedly administered at an interval between a first administration and a second administration, or between a second or further administration and another administration following said second or further administration (i.e., between consecutive administrations of the botulinum toxin) of between about 5 and 10 months, especially between about 6 and 9 months or between about 7 and 8 months.

Generally, the botulinum toxin used within the present invention is present in the form of a liquid composition. The liquid composition can be formulated by various techniques dependent on the desired application, as known in the art. It may be provided as a ready-to-use liquid formulation or in the form of a lyophilized powder that is to be reconstituted, typically in physiological saline, prior to use. Preferably, the botulinum toxin used within the present invention is in the form of an aqueous solution, more preferably a saline solution or a physiological saline solution, and most preferably a phosphate buffered physiological saline solution. The aqueous solution may additionally comprise one or more pharmaceutically acceptable substances. Suitable pharmaceutically acceptable substances comprise those well known in the art, see, e.g., Remington's Pharmaceutical Sciences, Mack Publishing Company, Easton, Pennsylvania.

In particular, the aqueous botulinum toxin solution or composition may include other carriers or non-toxic, non-therapeutic, non-immunogenic stabilizers and the like. Thus, the aqueous botulinum toxin composition may contain glycerol, protein stabilizers (HSA) or non-protein stabilizers such as polyvinyl pyrrolidone (PVP), hyaluronic acid or free amino acids, e.g., methionine or histidine. The aqueous botulinum toxin composition can be free of amino acids. In one embodiment, it may be free of stabilizing peptides (e.g., consisting of 5 to 50 amino acids, 10 to 40 amino acids or 15 to 30 amino acids). Suitable non-proteinaceous stabilizers for use herein are disclosed in WO 2005/007185 or WO 2006/020208. The botulinum toxin composition can also include non-ionic or ionic surfactant, e.g., polysorbate or poloxamer. A suitable formulation for HSA-stabilized formulation comprising a botulinum toxin according to the present invention is for example disclosed in U.S. Pat. No. 8,398,998 B2.

Preferably, the botulinum toxin used within the present invention is present in the form of an aqueous solution comprising sodium chloride (NaCl), more preferably in the form of a physiological saline solution (i.e. a solution including sodium chloride in physiological concentration, e.g., about 9 g/l NaCl), wherein the aqueous botulinum toxin solution comprises (i) no other excipient (except NaCl), (ii) human serum albumin (HSA) and a sugar, in particular a monosaccharide or a disaccharide, (iii) human serum albumin (HSA) and lactose, (iv) human serum albumin (HSA) and sucrose, (v) a monosaccharide and/or a disaccharide (e.g. lactose and/or sucrose), (vi) no buffer, (vii) no single amino acids, (viii) no human serum albumin (HSA), sodium chloride and lactose or no HSA, sodium chloride and sucrose, or (ix) no HSA and sodium chloride, or any combination of (i) to (ix).

In a second aspect, the present invention relates to a method for the rejuvenation of the neck and lower face by injecting botulinum toxin into platysma, wherein the botulinum toxin is injected in two to four platysma bands of a subject and in the platysma muscle at a jawline on both sides of the subject's face, wherein the botulinum toxin is injected in four to five injection points per platysma band and in three to four injection points in the platysma muscle at each jawline, and wherein the botulinum toxin is injected in the platysma bands at a dose of 3 U to 5 U and a volume of 0.04 ml to 0.06 ml per injection point and in the platysma muscle at each jawline at a dose of 3 U to 5 U and a volume of 0.04 ml to 0.06 ml per injection point.

This method of treatment is used for purely aesthetic purposes, i.e., for the rejuvenation of the neck and lower face and is therefore a cosmetic method. The botulinum toxin is generally intramuscularly injected using a suitable injection device, for example a syringe equipped with a 30 G, 32 G or 33 G needle. The syringe is not particularly limited and any conventional syringe may be used. A particularly suited syringe for use in accordance with the present invention is, for example, a 3Dose™ high precision syringe (vlow medical).

The location, number and spacing of injection points suited for used within the framework of the present invention are as disclosed herein above. An exemplary preferred injection pattern is shown in FIG. 2, according to which five injections spaced about 1 cm apart are made into the left and right medial and lateral bands, resulting in a total of 20 platysma band injections. In addition, two injections with a distance between injection points of about 2 cm are made in the left and right jawline, i.e., four injections in total. Following this exemplary injection pattern, an improvement in platysma bands prominence and sharpness of the jawline contour are obtained.

The method according to the second aspect of the present invention is closely related to the use according to the first aspect of the present invention. Thus, all definitions, explanations and advantages given herein with respect to the use according to the first aspect equally apply to the method according to the second aspect. In particular, the injection scheme and dosing regimen used in connection with the method according to the second aspect of the present invention is as disclosed herein above in relation to the use according to the first aspect of the present invention.

### EXAMPLES

### Example 1

### Intramuscular Injection of Dye in Porcine Muscle

A staining dye solution of 0.025% toluidine blue and 0.9% NaCl was injected into a pork loin muscle sample (porcine M. psoas major) using a vlow medical syringe equipped with a 30 G×½″ hypodermic needle. Three different injection volumes were used, i.e., 10 μl, 20 μl, and 40 μl, which were each injected in triplicate at 45° angle as well as at 90° angle and allowed to spread. Then, muscle pieces covering the lateral dye spread area were cut from the injected porcine M. psoas major and cross-sectional cuts thereof were also prepared.

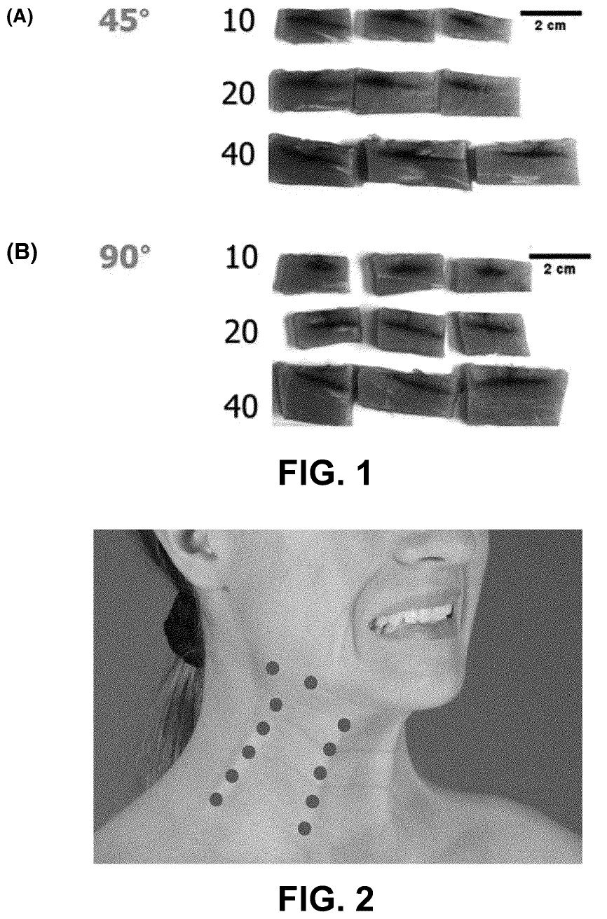

Photographs of the muscle cuts together with an indication of scale are shown in FIG. 1. The muscle cuts injected with 10 μl, 20 μl or 40 μl at 45° or 90° angle showing the lateral dye spread in top view are depicted in (A) and (B), respectively.

As can be seen from FIG. 1, the spread depends on the injection volume and occurs along the muscle fibers (even if perpendicular injected). The lateral spread of 10 μl and 40 μl toluidine dye solution at 90° injection angle was 10.4±2.2 mm and 23.3±5.4 mm, respectively.

These results, although obtained in an ex vivo preclinical model using porcine muscle, indicate that the injection of highly concentrated botulinum toxin at broadly spaced (e.g., 2 cm distance) injection points in the platysma muscle provides sufficient distribution for achieving the desired treatment effect even when injecting only small volumes of botulinum toxin.

### Example 2

### Injection Scheme and Dosing Regimen of the Present Invention

The botulinum toxin solution is administered using the high concentration (or “low volume-high dose”) approach underlying the present invention. Exemplary dosing regimens according to the present invention are shown in Table 1.

TABLE 1
Dosing regimens

| | | Jawlines | | | Platysma bands | | | |
|---|---|---|---|---|---|---|---|---|
| | | Dose/ | Volume/ | Total | Dose/ | Volume/ | Total | |
| Group | | injection | injection | Dose | injection | injection | Dose | Total |
| No. | Reconstitution | point | point | Jawline | point | point | Platysma | Dose |
| 1 | 200 U: 3.25 ml | 3.08 U | 0.05 ml | (2 × 2) × | 3.08 U | 0.05 ml | (4 × 5) × | 74 U |
| | 61.54 U/ml | | | 3.08 U = | | | 3.08 U = | |
| | | | | 12.3 U | | | 61.5 U | |
| 2 | 200 U: 2.5 ml | 4 U | 0.05 ml | (2 × 2) × | 4 U | 0.05 ml | (4 × 5) × | 96 U |
| | 80 U/ml | | | 4 U = | | | 4 U = | |
| | | | | 16 U | | | 80 U | |
| 3 | 200 U: 2.0 ml | 5 U | 0.05 ml | (2 × 2) × | 5 U | 0.05 ml | (4 × 5) × | 120 U |
| | 100 U/ml | | | 5 U = | | | 5 U = | |
| | | | | 20 U | | | 100 U | |

The injected botulinum toxin is typically a 0.9% saline solution. Any suitable injection device, such as a syringe equipped with a 32 G needle, may be used to administer the botulinum toxin solution by intramuscular injection in platysma, i.e., in platysma bands and in the platysma muscle at the jawlines.

With respect to the injection scheme, the botulinum toxin is preferably injected using an injection scheme as shown in FIG. 2. As can be seen, the injection scheme includes five injections spaced about 1 cm apart in each of the four platysma bands (lateral and medial bands on each side; 20 injections into platysma bands in total) and two injections spaced about 2 cm in the platysma muscle of the left and right jawline (4 injections in the jawlines; 24 injections in total for platysma bands and jawline).

Within the present invention, this injection scheme may also be modified to include only four injections spaced about 1-2 cm apart into four platysma bands (i.e., 16 injections in total in platysma bands) or, more preferably, five injections spaced about 1 cm in the left and right lateral bands and four injections in the left and right medial bands (i.e., 18 injections in total in platysma bands) or four injections spaced about 1-2 cm in the left and right lateral bands and five injections spaced about 1 cm in the left and right medial bands (i.e., 18 injections in total in platysma bands). Furthermore, the above-described modified injection schemes may further be modified in that they include three or four, rather than two, injections spaced about 1-2 cm, preferably 1.5 cm, in the platysma muscle of the left and right jawline (i.e., 6 or 8 injections in total in the jawlines).

1. (canceled)

2. The method of claim 15, wherein the botulinum toxin is injected at a dose of 4 U to 5 U per injection point in the platysma bands.

3. The method of claim 15, wherein the botulinum toxin is injected at a volume of 0.045 ml to 0.055 ml per injection point in the platysma bands.

4. The method of claim 15, wherein the botulinum toxin is injected at a dose of 4 U to 5 U per injection point in the platysma muscle at each jawline.

5. The method of claim 15, wherein the botulinum toxin is injected at a volume of 0.045 ml to 0.055 ml per injection point in the platysma muscle at each jawline.

6. The method of claim 15, wherein the botulinum toxin is injected at a concentration of 50 U/ml to 125 U/ml.

7. The method of claim 15, wherein the botulinum toxin is injected at a concentration of 60 U/ml to 120 U/ml er 70 U/ml to 110 U/ml.

8. The method of claim 15, wherein the botulinum toxin is injected in four platysma bands of a subject.

9. The method of claim 15, wherein the botulinum toxin is injected in five injection points per platysma band.

10. The method of claim 15, wherein the botulinum toxin is injected in two injection points in the platysma muscle at each jawline.

11. The method of claim 15, wherein the injection points along each platysma band are spaced at a distance of about 0.5 cm to about 2.0 cm.

12. The method of claim 15, wherein the injection points along each platysma band are spaced at a distance of about 0.5 cm to about 1.5 cm.

13. The method of claim 15, wherein the injection points along the platysma muscle at each jawline are spaced at a distance of about 0.5 cm to about 3.0 cm.

14. The method of claim 15, wherein the injection points along the platysma muscle at each jawline are spaced at a distance of about 1.5 cm to about 2.5 cm.

15. A method for the rejuvenation of the neck and lower face comprising:
injecting botulinum toxin in two to four platysma bands of a subject and in the platysma muscle at a jawline on both sides of the subject's face,
wherein the botulinum toxin is injected in four to five injection points per platysma band and in two to four injection points in the platysma muscle at each jawline, and wherein the botulinum toxin is injected in the platysma bands at a dose of 3 U to 5 U and a volume of 0.04 ml to 0.06 ml per injection point and in the platysma muscle at each jawline at a dose of 3 U to 5 U and a volume of 0.04 ml to 0.06 ml per injection point.

16. The method of claim 15, wherein the botulinum toxin is injected at a concentration of 70 U/ml to 110 U/ml.
